(19) United States
(12) Patent Application Publication
(10) Pub. No.: US 2024/0076319 A1
(43) Pub. Date: Mar. 7, 2024

(54) HELPER PLASMID AND METHOD FOR PREPARING RECOMBINANT ADENO-ASSOCIATED VIRUS

(71) Applicant: PACKGENE BIOTECH CO., LTD., Guangzhou (CN)

(72) Inventors: Huapeng Li, Guangzhou (CN); Jia Liu, Guangzhou (CN); Wanwen Xie, Guangzhou (CN)

(21) Appl. No.: 18/377,827

(22) Filed: Oct. 8, 2023

Related U.S. Application Data
(63) Continuation of application No. PCT/CN2022/078621, filed on Mar. 1, 2022.

(30) Foreign Application Priority Data
Apr. 8, 2021 (CN) 202110377907.X

(51) Int. Cl. C07K 14/005; C12N 7/00; C12N 15/63

(52) U.S. Cl. CPC C07K 14/005; C12N 7/00; C12N 15/63; C12N 2750/14122; C12N 2750/14143

(57) ABSTRACT

The present application provides a helper plasmid for preparing a recombinant adeno-associated virus (rAAV), including: 1) a coding sequence of an AAV Rep protein and a coding sequence of an AAV Cap protein; 2) at least one promoter sequence; and 3) at least one DA′sequence or AD′ sequence, wherein the AD′ sequence is a reverse complementary sequence of the DA′ sequence. The present application also provides a method for increasing a production capacity of a rAAV using the helper plasmid. With an AD′ sequence or a DA′ sequence, the helper plasmid provided in the present application can significantly increase an AAV yield of a cell.

### CROSS REFERENCE TO RELATED APPLICATION

The present application is a continuation application of PCT application No. PCT/CN2022/078621 filed on Mar. 1, 2022, which claims the benefit of Chinese Patent Application No. 202110377907.X filed on Apr. 8, 2021. The contents of all of the aforementioned applications are incorporated by reference herein in their entirety.

### REFERENCE TO SEQUENCE LISTING

The Sequence Listing XML file submitted via the USPTO Patent Center, with a file name of “Sequence_Listing.XML”, a creation date of Oct. 8, 2023, and a size of 14 KB, is part of the specification and is incorporated in its entirety by reference herein.

### TECHNICAL FIELD

The present application relates to genetic engineering and production of an adeno-associated virus (AAV) in a human cell, and specifically relates to a novel helper plasmid for AAV packaging and a use thereof in production of an AAV.

### BACKGROUND

Gene therapy is now becoming a reality from a dream of scientists forty years ago. After years of evolution and elimination of various viral and non-viral vectors, recombinant adeno-associated virus (rAAV) vectors are currently recognized as gene therapy vectors that meet the three criteria of safety, efficiency, and specificity. However, due to a limited capacity of large-scale production of AAV in the industry, the popularity of clinical applications of AAV is also restricted accordingly.

Therefore, it is urgent to break the bottleneck of a production capacity of AAV. The improvement and innovation of upstream production to increase an AAV yield is currently one of the key points to reduce a production cost for gene therapy. At present, a three-plasmid transfection method is widely used for production of AAV due to simplicity and rapidity. The three-plasmid transfection method involves co-transfection of HEK293 cells with three plasmids including: a helper plasmid pADhelper that provides four adenovirus elements; a helper plasmid pRC that provides coding sequences of AAV Rep and Cap proteins, where a Rep protein is responsible for replication of a genome of AAV and assistance in assembly of genomic particles of AAV, and a Cap protein is responsible for production of a capsid of AAV; and a target sequence-containing plasmid that can be referred to as pGOI, where a 5′ inverted terminal repeat (ITR) sequence and a 3′ ITR sequence in a natural genome of AAV are arranged upstream and downstream of a target sequence, respectively.

Typically, in a pRC plasmid for AAV serotype X (AAVX), a coding sequence is composed of Rep2 from AAV serotype 2 (AAV2) and CapX encoding a capsid protein of AAVX; and a P5 promoter is a key promoter for initiating the expression of rep and Cap, and the P5 promoter is usually arranged before a RepCap-coding region, as shown by “P5+RC” in FIG. 2. X. Xiao et al. have reported that the arrangement of a P5 promoter upstream and downstream of RC can increase a yield of AAV^{[1]}. In the target plasmid pGOI, ITRs arranged upstream and downstream of the target gene (or target sequence) each have a total length of 145 bp, which is considered to be related to the replication and guided packaging of a genome of AAV. Because there is an inverted self-complementary sequence in ITR, a T-shaped double-stranded structure will be formed through self-complementation in the case of single-stranded DNA. T. R. Flotte et al. have found that intact ITR has a promoter activity and can drive the expression of a CFTR gene^{[2]}. However, in terms of driving the gene expression, ITR only has a very low transcriptional activity compared with the traditional promoters^{[3]}.

### SUMMARY

In an aspect, the present application provides a helper plasmid for preparing a rAAV, including: 1) a coding sequence of an AAV Rep protein and a coding sequence of an AAV Cap protein; 2) at least one promoter sequence; and 3) at least one DA′ sequence or AD′ sequence, wherein the AD′ sequence is a reverse complementary sequence of the DA′ sequence.

In some embodiments, the at least one DA′ sequence or AD′ sequence is located upstream of the coding sequence of the AAV Rep protein and the coding sequence of the AAV Cap protein.

In some embodiments, the at least one DA′ sequence or AD′ sequence is located downstream of the coding sequence of the AAV Rep protein and the coding sequence of the AAV Cap protein.

In some embodiments, the helper plasmid includes sequentially from 5′ to 3′:
i) a DA′ sequence or an AD′ sequence, a promoter sequence, and a coding sequence of an AAV Rep protein and a coding sequence of an AAV Cap protein;
ii) a DA′ sequence or an AD′ sequence, a promoter sequence, a coding sequence of an AAV Rep protein and a coding sequence of an AAV Cap protein, and a DA′ sequence or an AD′ sequence;
iii) a DA′ sequence or an AD′ sequence, a promoter sequence, a coding sequence of an AAV Rep protein and a coding sequence of an AAV Cap protein, a DA′ sequence or an AD′ sequence, and a promoter sequence; or
iv) a coding sequence of an AAV Rep protein and a coding sequence of an AAV Cap protein, a DA′ sequence or an AD′ sequence, and a promoter sequence.

In some embodiments, a promoter is a P5 promoter.

In some embodiments, the P5 promoter sequence includes a sequence shown in SEQ ID NO: 2 or a functional variant having a sequence identity of at least 90% with the sequence shown in SEQ ID NO: 2.

In some embodiments, the DA′ sequence includes a sequence shown in SEQ ID NO: 4 or a functional variant having a sequence identity of at least 90% with the sequence shown in SEQ ID NO: 4.

In some embodiments, the AD′ sequence includes a sequence shown in SEQ ID NO: 3 or a functional variant having a sequence identity of at least 90% with the sequence shown in SEQ ID NO: 3.

In some embodiments, the AAV Rep protein and the AAV Cap protein are derived from AAV2.

In another aspect, the present application provides a method for increasing a production capacity of a rAAV, including: transforming a host cell with the helper plasmid described above.

The present application provides a helper and packaging plasmid for encoding Rep/Cap (including all serotypes), and with an AD′ sequence or a DA′ sequence, the helper and packaging plasmid can significantly increase an AAV yield of a single cell.

### BRIEF DESCRIPTION OF THE DRAWINGS

FIG. 1 is a schematic diagram of an ITR structure of a genome of AAV;

FIG. 2 is a schematic diagram of an experimental design to investigate functions of DA′ and AD in an embodiment; and

FIG. 3 shows results of comparison of a viral titer of a lysate of each plasmid tested in an embodiment with a control vector.

### DETAILED DESCRIPTION

Unless otherwise defined, all technical and scientific terms used herein have meanings commonly understood by those of ordinary skill in the art.

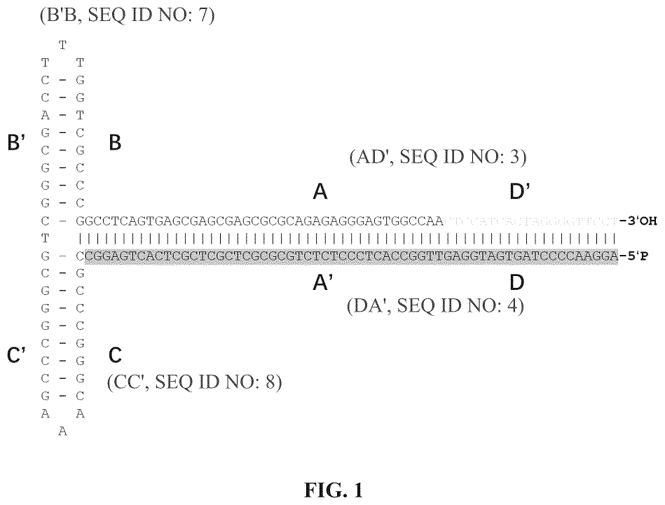

AAV: AAV was found first in adenovirus (AdV) preparations and later in human tissues. AAV belongs to the genus Dependoparvovirus in the family Parvoviridae, and is a defective virus with a simple structure. The replication of AAV requires the participation of a helper virus (such as an adenovirus). It is currently believed that AAV will not cause any human diseases, which is a basis for modifying AAV and using modified AAV in gene therapy. A genome of AAV is of about 4.7 kb and is a single-stranded DNA molecule, including two reading frames (rep and cap genes) and two ITRs at termini of the genome. The rep gene encodes a plurality of proteins for viral replication (Rep78, Rep68, Rep52, and Rep40), and the cap gene encodes three subunits of a capsid protein (VP1, VP2, and VP3). ITRs play a key role in the replication and packaging of AAV and are involved in the integration and escape of a viral genome on a genome of a host. ITR sequences (typically 145 bp) can form a T-shaped structure. As shown in FIG. 1, based on positions on the T-shaped structure, the ITR sequences can be divided into various segments such as A, B, B′, C′, C, A′, and D. On the ITR sequences, A and A′ are complementary, B and B′ are complementary, C and C′ are complementary, and D and D′ are complementary. Structurally, the ITR sequences include two palindrome arms (B′-B (SEQ ID NO: 7, 5′-cgggcgacctttggtcgcccgg-3′) and C-C′ (SEQ ID NO: 8, 5′-cgcccgggcaaagcccgggcg-3′)) and a long-stemmed palindrome (A′-A). The segment D′ is located in an ITR sequence at a 5′ terminus of the genome of AAV, and the segment D is located in an ITR sequence at a 3′ terminus of the genome of AAV.

rAAV: rAAV refers to an AAV obtained by modifying a genome of a wild-type (WT) AAV. rAAV typically has a capsid protein and structure of WT AAV, but the protein-coding sequences (rep and cap genes) in a genome of WT AAV are replaced by a target gene (such as a therapeutic gene), and only the ITR sequences at two termini are retained. An important role of rAAV is to serve as a vector for gene therapy.

Helper plasmid: The helper plasmid is a helper plasmid for preparing rAAV. To prepare rAAV, a host cell (such as a mammalian or insect cell) generally needs to be co-transfected with three plasmids: a helper plasmid pADhelper that provides four adenovirus elements; a helper plasmid pRC that provides coding sequences of AAV Rep and Cap proteins, where a Rep protein is responsible for replication of a genome of AAV and assistance in assembly of genomic particles of AAV, and a Cap protein is responsible for production of a capsid of AAV; and a target sequence (such as a therapeutic gene)-containing plasmid that can be referred to as pGOI.

AD′ sequence: The AD′ sequence refers to a sequence formed through the combination of segments A and D′ described above. In a specific embodiment, the AD′ sequence is a sequence shown in SEQ ID NO: 3.

DA′ sequence: The DA′ sequence refers to a sequence formed through the combination of segments D and A′ described above. In a specific embodiment, the DA′ sequence is a sequence shown in SEQ ID NO: 4.

In the embodiments described herein, unless otherwise indicated, the AD′ sequence and the DA′ sequence may be used interchangeably, for example, when the AD′ sequence is mentioned, it should be understood that the DA′ sequence is also mentioned.

Sequence identity: When a nucleotide sequence is mentioned, the term “sequence identity” refers to a degree of consistency between two nucleotide sequences (such as a query sequence and a reference sequence), and is generally expressed in a percentage. In general, before a percentage of consistency between two nucleotide sequences is calculated, sequence alignment is conducted and a gap (if any) is introduced. If bases in two sequences at a specified alignment position are the same, the two sequences are considered to be consistent or matched at this position; and if bases in two sequences at a specified alignment position are different, the two sequences are considered to be inconsistent or mismatched at this position. In some algorithms, a number of matched positions are divided by a total number of positions in an alignment window to obtain a sequence identity. In some other algorithms, a number of gaps and/or a length of gaps are also taken into account. For the purpose of the present application, the public alignment software BLAST (available on the website: ncbi.nlm.ih.gov) may be adopted, and with default settings, the optimal sequence alignment is allowed and a sequence identity between two nucleotide sequences is calculated.

Functional variant: The functional variant refers to a nucleic acid obtained through a variation of at least one nucleotide (including replacement, addition, or deletion) in a basic nucleic acid, but the nucleic acid obtained after the variation basically retains the original biological functions. After exemplary nucleic acid sequences are given, those skilled in the art can seek to obtain functional variants of these nucleic acid sequences through conventional experimental methods. Thus, in some embodiments, the P5 promoter sequence included in the pRC helper plasmid provided herein may include a sequence shown in SEQ ID NO: 2. In some other embodiments, the P5 promoter sequence may be a functional variant having a sequence identity of at least 90% (such as at least 95% or at least 98%) with the sequence shown in SEQ ID NO: 2. In some embodiments, the DA′ sequence included in the pRC helper plasmid provided herein may include a sequence shown in SEQ ID NO: 4. In some other embodiments, the DA′ sequence is a functional variant having a sequence identity of at least 90% (such as at least 92%, at least 95%, or at least 98%) with the sequence shown in SEQ ID NO: 4. In some embodiments, the AD′ sequence included in the pRC helper plasmid provided herein may include a sequence shown in SEQ ID NO: 3. In some other embodiments, the AD′ sequence is a functional variant having a sequence identity of at least 90% (such as at least 92%, at least 95%, or at least 98%) with the sequence shown in SEQ ID NO: 3.

All pRC-series vectors involved in the present application are obtained by a conventional molecular cloning method.

In the pRC helper plasmid provided herein, the P5 promoter is used in combination with the DA′ sequence or AD′ sequence, which can significantly improve a yield of rAAV.

The present application is further elaborated below through specific examples. In the present application, unless otherwise specified, a concentration of a solid or liquid reagent refers to a mass concentration, and a concentration of a gas refers to a volume concentration.

### Examples

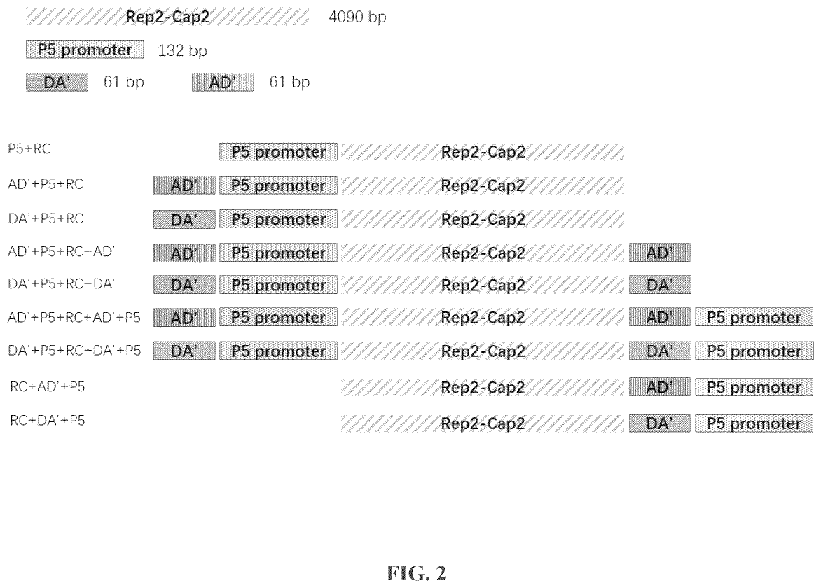

By designing combinations containing various numbers and arrangement positions of DA′ or AD′ sequences and P5 promoters, different candidate helper plasmids containing Rep2Cap2 were constructed based on a backbone vector (SEQ ID NO: 1) by a conventional molecular cloning method (FIG. 2), and then virus-producing titers of the helper plasmids in a common three-plasmid transfection system were compared in parallel.

Brief steps of three-plasmid transfection to produce AAV:

1) About 3×10⁵ of 293T cells (293T, derived from ATCC® CRL-3216™) were inoculated into a 24-well plate, a high-sugar DMEM medium including 10% fetal bovine serum (FBS), 2 mM L-glutamine (ATCC 30-2214), and 1% Penicillin/Streptomycin was added, and the cells were cultivated at 37° C. and 5% CO₂ for about 16 h, where a cell density during transfection was about 60% to 70%.

2) pRC and pAdHleper helper vectors (vector source: UPENN vector core PL-F-PVADF6, the University of Pennsylvania) and an AAV plasmid containing the fluorescent protein gene driven by a CAG promoter (pAAV.CAG.EGFP) were added to 0.5 mL of DMEM according to a ratio of 0.5 μg:0.5 μg:0.5 μg, then 3 μL of polyethylenimine (PEI) (1 μg/μL) was added, and a resulting mixture was immediately mixed and placed at room temperature for 10 min to obtain a mixed medium for transfection; and the medium in the 24-well plate was removed, the mixed medium for transfection was added, and the cells were further cultivated in a 37° C. incubator (5% CO₂ concentration).

3) After the cells were further cultivated for 72 h, cells and supernatant were collected, repeatedly frozen and thawed (thawing and freezing each were conducted for 2 min) three times in a 37° C. water bath and a dry ice-ethanol bath, respectively, and then centrifuged at 10,000 g for 10 min to obtain a supernatant, which was an AAV crude extract.

A method for titer determination of AAV:

Sequences of ITRs of all types of AAV vectors were specifically detected using primers

FWD ITR
(SEQ ID NO: 5)
(5′-GGAACCCCTAGTGATGGAGTT)
and
REV ITR
(SEQ ID NO: 6)
(5′-CGGCCTCAGTGAGCGA)

(1) Digestion of a Sample by Dnase I

Components of a reaction system were listed in the table below.

| Reagent | X1 | |
|---|---|---|
| Nuclease-free Water | 15.8 | μL |
| 10x DNase Buffer | 2 | μL |
| DNase I, RNase free l (70 U/μL) | 0.2 | μL |
| (purchased from Roche) | | |
| Sample | 2 | μL |

5 μL of a sample was taken and diluted 20-fold; a corresponding number of PCR tubes were prepared; 18 μL of a digestion solution was added to each tube, and then 2 μL of a diluted sample and 2 μL of a plasmid standard (including 4×10⁸ of AAV copies (Genome Copies, GC) as a DNase digestion control) for plotting a standard curve, where the diluted sample was further diluted 10-fold; the tubes were incubated at 37° C. for 30 min; and after digestion was completed, 5 μL of a sample was taken and added to 95 μL of water, and the dilution was conducted 2 times serially until the sample was diluted 80,000-fold, but RefAAV (plasmid standard) was serially diluted 4,000-fold.

(2) SYBR GreenqPCR

Standard preparation: A plasmid standard including 2×10⁸ of AAV copies/μL was taken and subjected to 6 serial dilutions with 8 μL of standard+72 μL of water. A first gradient concentration was 2×10⁸ GC/μL, and it was set to 8×10¹⁴ GC/mL in software to reflect a dilution gradient of a sample. Subsequent gradient concentrations were 8×10¹³ GC/mL, 8×10¹² GC/mL, 8×10¹¹ GC/mL, 8×10¹⁰ GC/mL, and 8×10⁹ GC/mL, respectively.

Components of a reaction system were listed in the table below.

| Reagent | Vol. per reaction | |
|---|---|---|
| SYBR PCR reagent (2X) | 10 | μL |
| ROX (50X) | 0.4 | μL |
| FWD ITR (50 μM) | 0.1 | μL |
| REV ITR (50 μM) | 0.1 | μL |
| Nuclease-free water | 4.4 | μL |
| Sample DNA | 5 | μL |

With 3 replicate wells per sample, a corresponding volume of a mixture was prepared, 18 μL of the mixture was added per well, and then 2 μL of a sample was added per well.

(3) SYBR Green qPCR Conditions

Pre-denaturation: 95° C. for 10 min;

Cycling: 40 cycles: 95° C. for 15 s, and 60° C. for 1 min.

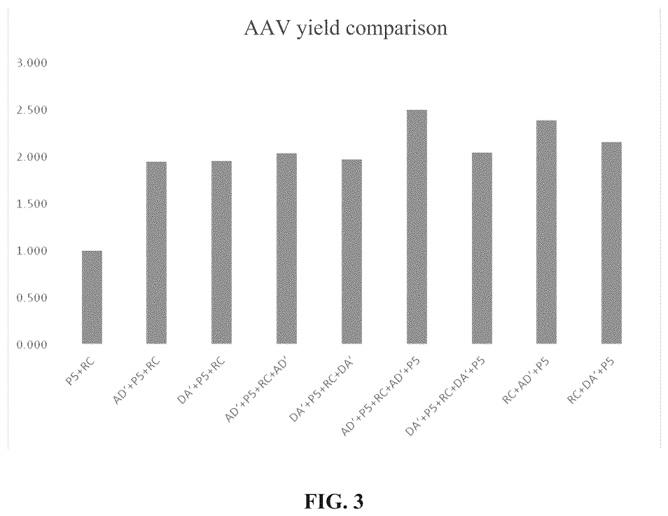

All titer test results were converted into fold relationships relative to a control vector P5+RC, and the fold relationships were shown in FIG. 3. The results show that the helper plasmid containing a DA′ sequence or an AD′ sequence has an effect of increasing a yield of rAAV compared with Rep2Cap2 vector “P5+RC” driven by a single P5 promoter.

A helper plasmid of a specific serotype (Rep2Cap2) is used in the examples. Those skilled in the art should understand that the present application is not limited to this specific serotype, but helper plasmids of other serotypes currently known and to be found in the future may be used to implement the present application.

Some of the nucleic acid sequences mentioned herein are as follows:

Plasmid backbone sequence (SEQ ID NO: 1):
aagccgaattctgcagatatccatcacactggcggccgct
cgactagagcggccgccaccgcggtggagctccagctttt
gttccctttagtgagggttaattgcgcgcttggcgtaatc
atggtcatagctgtttcctgtgtgaaattgttatccgctc
acaattccacacaacatacgagccggaagcataaagtgta
aagcctggggtgcctaatgagtgagctaactcacattaat
tgcgttgcgctcactgcccgctttccagtcgggaaacctg
tcgtgccagctgcattaatgaatcggccaacgcgggggag
aggcggtttgcgtattgggcgctcttccgcttcctcgctc
actgactcgctgcgctcggtcgttcggctgcggcgagcgg
tatcagctcactcaaaggcggtaatacggttatccacaga
atcaggggataacgcaggaaagaacatgtgagcaaaaggc
cagcaaaaggccaggaaccgtaaaaaggccgcgttgctgg
cgtttttccataggctccgcccccctgacgagcatcacaa
aaatcgacgctcaagtcagaggtggcgaaacccgacagga
ctataaagataccaggcgtttccccctggaagctccctcg
tgcgctctcctgttccgaccctgccgcttaccggatacct
gtccgcctttctcccttcgggaagcgtggcgctttctcat
agctcacgctgtaggtatctcagttcggtgtaggtcgttc
gctccaagctgggctgtgtgcacgaaccccccgttcagcc
cgaccgctgcgccttatccggtaactatcgtcttgagtcc
aacccggtaagacacgacttatcgccactggcagcagcca
ctggtaacaggattagcagagcgaggtatgtaggcggtgc
tacagagttcttgaagtggtggcctaactacggctacact
agaagaacagtatttggtatctgcgctctgctgaagccag
ttaccttcggaaaaagagttggtagctcttgatccggcaa
acaaaccaccgctggtagcggtggtttttttgtttgcaag
cagcagattacgcgcagaaaaaaaggatctcaagaagatc
ctttgatcttttctacggggtctgacgctcagtggaacga
aaactcacgttaagggattttggtcatgagattatcaaaa
aggatcttcacctagatccttttaaattaaaaatgaagtt
ttaaatcaatctaaagtatatatgagtaaacttggtctga
cagttaccaatgcttaatcagtgaggcacctatctcagcg
atctgtctatttcgttcatccatagttgcctgactccccg
tcgtgtagataactacgatacgggagggcttaccatctgg
ccccagtgctgcaatgataccgcgagacccacgctcaccg
gctccagatttatcagcaataaaccagccagccggaaggg
ccgagcgcagaagtggtcctgcaactttatccgcctccat
ccagtctattaattgttgccgggaagctagagtaagtagt
tcgccagttaatagtttgcgcaacgttgttgccattgcta
caggcatcgtggtgtcacgctcgtcgtttggtatggcttc
attcagctccggttcccaacgatcaaggcgagttacatga
tcccccatgttgtgcaaaaaagcggttagctccttcggtc
ctccgatcgttgtcagaagtaagttggccgcagtgttatc
actcatggttatggcagcactgcataattctcttactgtc
atgccatccgtaagatgcttttctgtgactggtgagtact
caaccaagtcattctgagaatagtgtatgcggcgaccgag
ttgctcttgcccggcgtcaatacgggataataccgcgcca
catagcagaactttaaaagtgctcatcattggaaaacgtt
cttcggggcgaaaactctcaaggatcttaccgctgttgag
atccagttcgatgtaacccactcgtgcacccaactgatct
tcagcatcttttactttcaccagcgtttctgggtgagcaa
aaacaggaaggcaaaatgccgcaaaaaagggaataagggc
gacacggaaatgttgaatactcatactcttcctttttcaa
tattattgaagcatttatcagggttattgtctcatgagcg
gatacatatttgaatgtatttagaaaaataaacaaatagg
ggttccgcgcacatttccccgaaaagtgccacctaaattg
taagcgttaatattttgttaaaattcgcgttaaatttttg
ttaaatcagctcattttttaaccaataggccgaaatcggc
aaaatcccttataaatcaaaagaatagaccgagatagggt
tgagtgttgttccagtttggaacaagagtccactattaaa
gaacgtggactccaacgtcaaagggcgaaaaaccgtctat
cagggcgatggcccactacgtgaaccatcaccctaatcaa
gttttttggggtcgaggtgccgtaaagcactaaatcggaa
ccctaaagggagcccccgatttagagcttgacggggaaag
ccggcgaacgtggcgagaaaggaagggaagaaagcgaaag
gagcgggcgctagggcgctggcaagtgtagcggtcacgct
gcgcgtaaccaccacacccgccgcgcttaatgcgccgcta
cagggcgcgtcccattcgccattcaggctgcgcaactgtt
gggaagggcgatcggtgcgggcctcttcgctattacgcca
gctggcgaaagggggatgtgctgcaaggcgattaagttgg
gtaacgccagggttttcccagtcacgacgttgtaaaacga
cggccagtgagcgcgcgtaatacgactcactatagggcga
attgggtaccgggccccccctcgatcgaggtcgacggtat
cgggggagct
P5 promoter sequence (SEQ ID NO: 2):
aggtcctgtattagaggtcacgtgagtgttttgcgacat
tttgcgacaccatgtggtcacgctgggtatttaagcccga
gtgagcacgcagggtctccattttgaagcgggaggtttga
acgcgcagccgcc
AD′ sequence (SEQ ID NO: 3):
gcctcagtgagcgagcgagcgcgcagagagggagtggcc
aactccatcactaggggttcct
DA′ sequence (SEQ ID NO: 4):
aggaacccctagtgatggagttggccactccctctctgc
gcgctcgctcgctcactgaggc

### CITED REFERENCES

1. Xiao X, Li J, Samulski R J. Production of high-titer recombinant adeno-associated virus vectors in the absence of helper adenovirus. J Virol. 1998 March; 72(3):2224-32. doi: /JVI.72.3.2224-2232.1998. PMID: 9499080; PMCID: PMC109519.
2. Flotte T R, Afione S A, Solow R, Drumm M L, Markakis D, Guggino W B, Zeitlin P L, Carter B J. Expression of the cystic fibrosis transmembrane conductance regulator from a novel adeno-associated virus promoter. J Biol Chem. 1993 Feb. 15; 268(5):3781-90. PMID: 7679117.
3. Zhang L, Wang D H, Fischer H, Fan P D, Widdicombe J H, Kan Y W, Dong J Y. Efficient expression of CFTR function with adeno-associated virus vectors that carry shortened CFTR genes.PNAS.1998 Aug. 18; 95 (17) 10158-10163.

1. A helper plasmid for preparing a recombinant adeno-associated virus (rAAV), comprising:
1) a coding sequence of an AAV Rep protein and a coding sequence of an AAV Cap protein;
2) at least one promoter sequence; and
3) at least one DA′ sequence or AD′ sequence, wherein the AD′ sequence is a reverse complementary sequence of the DA′ sequence.

2. The helper plasmid according to claim 1, wherein the at least one DA′ sequence or AD′ sequence is located upstream of the coding sequence of the AAV Rep protein and the coding sequence of the AAV Cap protein.

3. The helper plasmid according to claim 1, wherein the at least one DA′ sequence or AD′ sequence is located downstream of the coding sequence of the AAV Rep protein and the coding sequence of the AAV Cap protein.

4. The helper plasmid according to claim 2, wherein the at least one DA′ sequence or AD′ sequence is located downstream of the coding sequence of the AAV Rep protein and the coding sequence of the AAV Cap protein.

5. The helper plasmid according to claim 1, wherein the helper plasmid comprises sequentially from 5′ to 3′:
i) a DA′ sequence or an AD′ sequence, a promoter sequence, and a coding sequence of an AAV Rep protein and a coding sequence of an AAV Cap protein;
ii) a DA′ sequence or an AD′ sequence, a promoter sequence, a coding sequence of an AAV Rep protein and a coding sequence of an AAV Cap protein, and a DA′ sequence or an AD′ sequence;
iii) a DA′ sequence or an AD′ sequence, a promoter sequence, a coding sequence of an AAV Rep protein and a coding sequence of an AAV Cap protein, a DA′ sequence or an AD′ sequence, and a promoter sequence; or
iv) a coding sequence of an AAV Rep protein and a coding sequence of an AAV Cap protein, a DA′ sequence or an AD′ sequence, and a promoter sequence.

6. The helper plasmid according to claim 2, wherein the helper plasmid comprises sequentially from 5′ to 3′:
i) a DA′ sequence or an AD′ sequence, a promoter sequence, and a coding sequence of an AAV Rep protein and a coding sequence of an AAV Cap protein;
ii) a DA′ sequence or an AD′ sequence, a promoter sequence, a coding sequence of an AAV Rep protein and a coding sequence of an AAV Cap protein, and a DA′ sequence or an AD′ sequence; or
iii) a DA′ sequence or an AD′ sequence, a promoter sequence, a coding sequence of an AAV Rep protein and a coding sequence of an AAV Cap protein, a DA′ sequence or an AD′ sequence, and a promoter sequence.

7. The helper plasmid according to claim 3, wherein the helper plasmid comprises sequentially from 5′ to 3′:
i) a DA′ sequence or an AD′ sequence, a promoter sequence, a coding sequence of an AAV Rep protein and a coding sequence of an AAV Cap protein, and a DA′ sequence or an AD′ sequence;
ii) a DA′ sequence or an AD′ sequence, a promoter sequence, a coding sequence of an AAV Rep protein and a coding sequence of an AAV Cap protein, a DA′ sequence or an AD′ sequence, and a promoter sequence; or
iii) a coding sequence of an AAV Rep protein and a coding sequence of an AAV Cap protein, a DA′ sequence or an AD′ sequence, and a promoter sequence.

8. The helper plasmid according to claim 1, wherein a promoter is a P5 promoter.

9. The helper plasmid according to claim 5, wherein a promoter is a P5 promoter.

10. The helper plasmid according to claim 1, wherein the promoter sequence comprises a sequence shown in SEQ ID NO: 2 or a functional variant having a sequence identity of at least 90% with the sequence shown in SEQ ID NO: 2.

11. The helper plasmid according to claim 2, wherein the promoter sequence comprises a sequence shown in SEQ ID NO: 2 or a functional variant having a sequence identity of at least 90% with the sequence shown in SEQ ID NO. 2.

12. The helper plasmid according to claim 5, wherein the promoter sequence comprises a sequence shown in SEQ ID NO: 2 or a functional variant having a sequence identity of at least 90% with the sequence shown in SEQ ID NO: 2.

13. The helper plasmid according to claim 1, wherein the DA′ sequence comprises a sequence shown in SEQ ID NO: 4 or a functional variant having a sequence identity of at least 90% with the sequence shown in SEQ ID NO: 4.

14. The helper plasmid according to claim 5, wherein the DA′ sequence comprises a sequence shown in SEQ ID NO: 4 or a functional variant having a sequence identity of at least 90% with the sequence shown in SEQ ID NO: 4.

15. The helper plasmid according to claim 1, wherein the AD′ sequence comprises a sequence shown in SEQ ID NO: 3 or a functional variant having a sequence identity of at least 90% with the sequence shown in SEQ ID NO: 3.

16. The helper plasmid according to claim 5, wherein the AD′ sequence comprises a sequence shown in SEQ ID NO: 3 or a functional variant having a sequence identity of at least 90% with the sequence shown in SEQ ID NO: 3.

17. The helper plasmid according to claim 1, wherein the AAV Rep protein and the AAV Cap protein are derived from adeno-associated virus serotype 2 (AAV2).

18. The helper plasmid according to claim 5, wherein the AAV Rep protein and the AAV Cap protein are derived from AAV2.

19. A method for increasing a production capacity of a rAAV, comprising: transforming a host cell with the helper plasmid according to claim 1.

20. A method for increasing a production capacity of a rAAV, comprising: transforming a host cell with the helper plasmid according to claim 5.


SEQUENCE LISTING (8 sequences)

SEQ ID NO: 1 - DNA, 2971 bp, synthetic construct
  source 1..2971: mol_type = other DNA
  misc_feature 1..2971: note = synthetic
aagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagct

SEQ ID NO: 2 - DNA, 132 bp, synthetic construct
  source 1..132: mol_type = other DNA
  misc_feature 1..132: note = synthetic
aggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 3 - DNA, 61 bp, synthetic construct
  source 1..61: mol_type = other DNA
  misc_feature 1..61: note = synthetic
gcctcagtgagcgagcgagcgcgcagagagggagtggccaactccatcactaggggttcct

SEQ ID NO: 4 - DNA, 61 bp, synthetic construct
  source 1..61: mol_type = other DNA
  misc_feature 1..61: note = synthetic
aggaacccctagtgatggagttggccactccctctctgcgcgctcgctcgctcactgaggc

SEQ ID NO: 5 - DNA, 21 bp, synthetic construct
  source 1..21: mol_type = other DNA
  misc_feature 1..21: note = synthetic
ggaacccctagtgatggagtt

SEQ ID NO: 6 - DNA, 16 bp, synthetic construct
  source 1..16: mol_type = other DNA
  misc_feature 1..16: note = synthetic
cggcctcagtgagcga

SEQ ID NO: 7 - DNA, 22 bp, synthetic construct
  source 1..22: mol_type = other DNA
  misc_feature 1..22: note = synthetic
cgggcgacctttggtcgcccgg

SEQ ID NO: 8 - DNA, 21 bp, synthetic construct
  source 1..21: mol_type = other DNA
  misc_feature 1..21: note = synthetic
cgcccgggcaaagcccgggcg